(19) United States
(12) Patent Application Publication
(10) Pub. No.: US 2015/0007528 A1
(43) Pub. Date: Jan. 8, 2015

(54) Strap system for annimals to enable positioning of pouch holding cooling or warming medium in a targeted area of the chest

(71) Applicant: Susan Marie Landrith, Boca Raton, FL (US)

(72) Inventor: Susan Marie Landrith, Boca Raton, FL (US)

(21) Appl. No.: 13/934,313

(22) Filed: Jul. 3, 2013

(51) Int. Cl. A01K 13/00; B68B 5/00

(52) U.S. Cl. CPC A01K 13/003; B68B 5/00
USPC 54/68

(57) ABSTRACT

It is sometimes desirable to apply and hold medication, ice or warming medium against the chest wall of an animal (i.e. an ice pack). Due of the anatomy of animals, this is difficult to do remotely without holding the medium against the chest wall with a hand or adhesive tape. My invention provides a way to attach a pouch or pack to the device and then, when the device is correctly placed over the animal's head and secured at the girth, firmly, but gently holds the medium in the targeted place on the chest wall of the animal.

### CROSS REFERENCE TO RELATED APPLICATIONS

This application is a utility patent application.

### BACKGROUND OF THE INVENTION

### Field of the Invention

This invention pertains generally to animal husbandry and/or pets generally.

There are instances where it may be desirable to be able to remotely apply medication, cooling or warming medium to a targeted area of the chest in animals. The anatomy of an animal makes it difficult to keep, for example, an ice pack firmly against the chest wall of the animal without the need to physically apply the pressure needed to hold the ice pack against the chest wall.

My device solves this problem with a system of straps that has a means to affix a medium containing medication, ice or warming medium, applies gentle pressure where needed on the chest wall, is comfortable for the animal, is cost effective to manufacture and is simple to use.

### DESCRIPTION

The device is comprised of the following (please refer to FIGS. 3 and 4 on the drawings at the end of this application):

One strap of elastic 72 inches long by 1½ inches wide (Item No. 10);

One strap of nylon propylene 6½ inches long by 1½ inches wide (Item No. 12);

One strap of nylon propylene 43 inches long by 1½ inches (Item No. 13)

Several strips of securement loops (Item Nos. 11, 14, 15, 16, 17, 18 and 20).

7 inch by 7 inch synthetic lamb's wool

All elements of the devise are necessary to comfortable hold a pack or pouch containing the medication, cooling or warming medium against the chest wall of the animal.

### DETAILED DESCRIPTION OF THE DEVICE

Take Item 10 and stretch it out on a flat surface. Sew the right end of Item 10 to the right end of Item 12 so that Item 10 is at a right angle to Item 12 (FIG. 1). Sew a 1 inch by 1 inch piece of securement loop (hook side) (Item 17) to the top side of Item 10 at the left end (FIG. 1). Sew a 1 in by 1 inch piece of securement loop (soft side) (Item 18) to the left end of Item 12 on the back of Item 12 (FIG. 1). Sew one 3½ inch by 1 inch piece of securement loop on the front of Item 10, 8½ inches above the line where the top of Item 12 rests on Item 10 on each end (FIG. 3). Sew a 1 inch by 1 inch piece of securement loop (hook side) (Item 16) to the right end of Item 13 (FIG. 2). Sew Item 13, with the securement loop to Item 12, on the back side, in the center of Item 12, making a “T” shape with Item 12 and Item 13 (FIG. 2). Turn the strap system over so you are looking at the Front View (FIG. 3). Sew a 3 inch long by 1 inch wide securement loop strip (hook side) (Item 15) to the left end of Item 13 (FIG. 3). Sew a 12 inch piece of securement loop (soft side) (Item 14) thirteen inches away from the end of Item 15 (FIG. 3). Take the synthetic lamb's wool (Item 19) and sew a 1 inch by 1 inch piece of securement loop (soft side) (Item 20) three inches from the left side and one-fourth inch from the bottom (FIG. 6). Take synthetic lamb's wool (Item 19) and with right sides together; sew the synthetic lamb's wool (Item 19) on three sides with a ⅝″ seam. Turn synthetic lamb's wool, as sewn, right side out. Fold the raw edges on the open end in to form a neat seam and stitch. Attach the synthetic lamb's wool (Item 19) to the strap system by placing the securement loop on the synthetic lamb's wool (Item 20) against the securement loop (Item 16) on the device. The finished device is shown in FIGS. 3, 4, 5 and 6.

Varying lengths and widths of nylon and propylene to accommodate Nos. 10 and 13 can be used for larger or small animals.

The device is used by first, detaching Item 10 from Item 12 at the securement loops (FIG. 2). Insert the end of Item 10 into the sleeve of the pack or pouch (like pouch published under US 2011/0061607 A1, for example. The pouch or pack could have tabs with securement loops to provide size adjustments. Reattach Item 10 to Item 12 with the securement loops. Place the elastic portion (Item 10) around the neck of the animal with the lamb's wool against the skin of the animal (to prevent any abrasions form the device) (FIG. 7). Adjust location of the pack or pouch to target the particular area of the chest desired. Place Item 13 between the legs of animal and loop around a saddle cinch strap or a girth strap (not a part of this claim) and attach the securement loops at the end of Item 13 to the longer strip of securement loops (Item 14). The pouch or pack is now remotely held against the targeted chest area of the animal. See FIG. 7 for device placement.

### DRAWINGS

The attached drawings consist of the following:

FIG. 1 shows the front view of the portion of the strap assembly that is placed over the animal's neck.

FIG. 2 shows the back view of the strap system and demonstrates where the neck strap (FIG. 10) detach from the smaller strap section labels as FIG. 12.

FIG. 3 shows the front view of the strap system

FIG. 4 shows the back view of the strap system in its fully attached state.

FIG. 5 shows the side view of the strap system.

FIG. 6 shows how padding can be attached to protect against skin irritation where the strap system comes in contact with a potentially sensitive location of an animal's body.

FIG. 7 shows how the device would look on a horse.

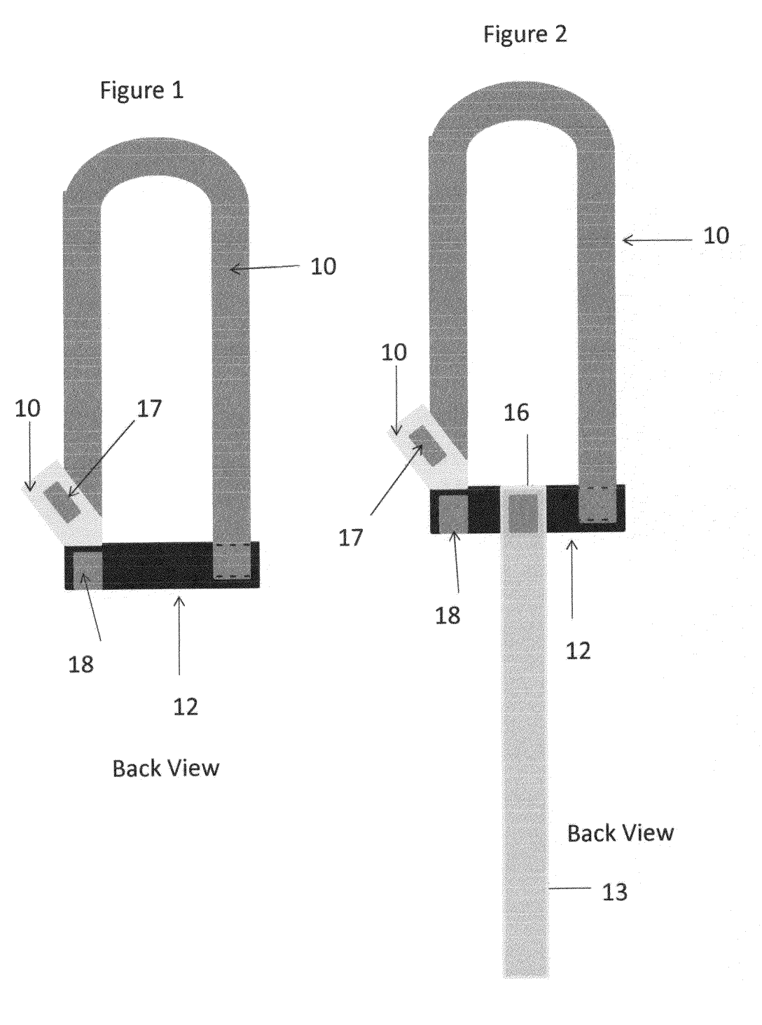

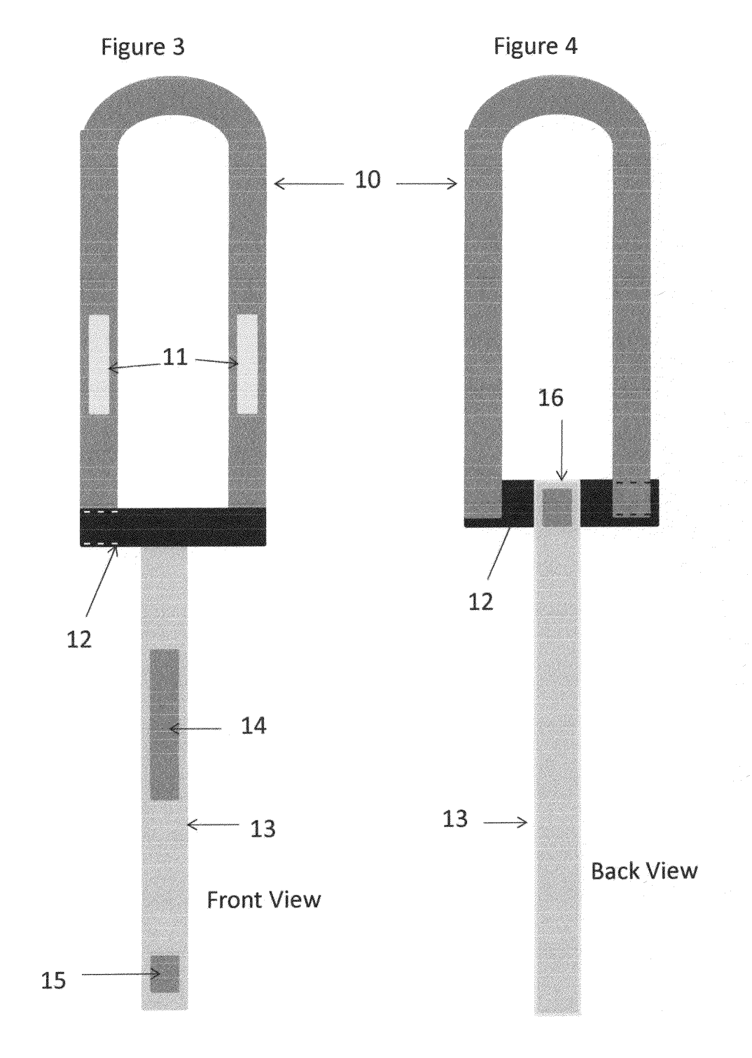

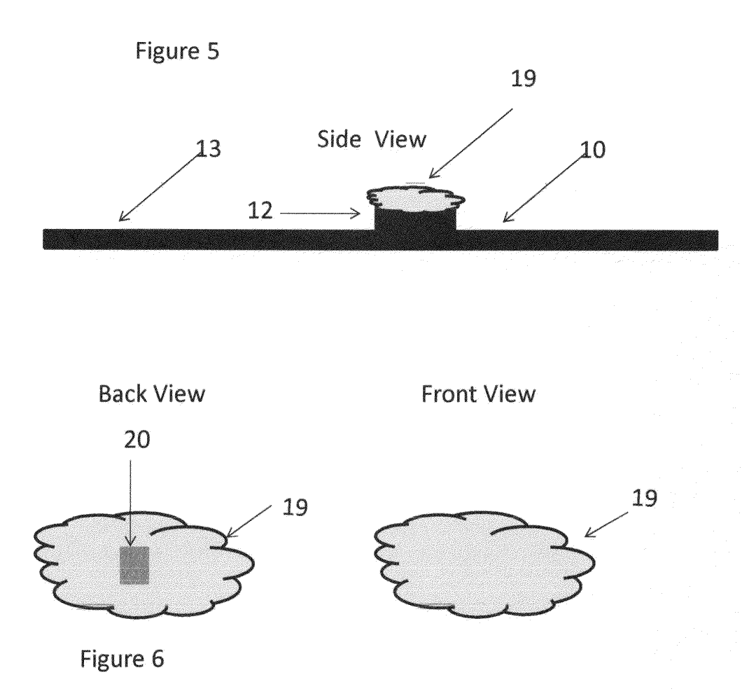

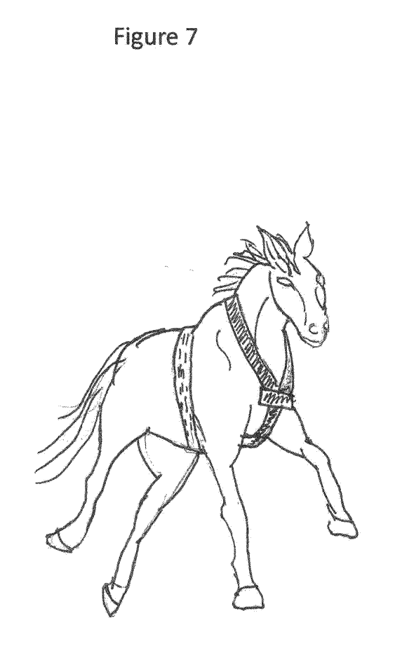

1. I claim as my invention a device that when properly placed on an animal, will allow a pouch or pack of medication, ice or warming medium to be held remotely against the chest wall of an animal comprising in combination:
an elastic strap having securement loops attached to a strap at a right angle with securement loops.
With a further strap attached at a center right angle to the previous strap having securement loops
With a soft pad having securement loops;

2. The elastic strap of claim 1 with buckles attached on each end so as to facilitate attachment to the further nylon strap of claim 1.

3. The nylon strap of claim 1 consisting of two nylon pieces joined together to form a “T” shape containing buckles on each end of the strap forming the top of the “T” shape to facilitate attachment to the elastic strap of claim 1.
